(19) United States
(12) Patent Application Publication
(10) Pub. No.: US 2015/0038830 A1
(43) Pub. Date: Feb. 5, 2015

(54) MAGNETIC RESONANCE DEVICE

(71) Applicant: SIEMENS AKTIENGESELLSCHAFT, MUNCHEN (DE)

(72) Inventors: MATHIAS BLASCHE, BUCKENHOF (DE); THORSTEN FEIWEIER, POXDORF (DE); MATTHIAS GEBHARDT, ERLANGEN (DE); MARCEL KRUIP, OXFORD (GB); KARSTEN WICKLOW, BAMBERG (DE)

(21) Appl. No.: 14/296,481

(22) Filed: Jun. 5, 2014

(30) Foreign Application Priority Data
Jul. 30, 2013 (DE) 102013214880.1

(51) Int. Cl. G01R 33/385; A61B 5/055; G01R 33/48

(52) U.S. Cl. CPC G01R 33/385; G01R 33/48; A61B 5/0555
USPC 600/415; 324/322

(57) ABSTRACT

A magnetic resonance device with at least one main field magnet and at least one gradient coil is provided. The magnetic resonance device provides more free space for the patient and treatment providers; the magnetic resonance device has at least one main field magnet, which is provided to generate a magnet volume. The magnet volume has an imaging region, which is displaced from the center of the main field magnet along a longitudinal axis defined by the main field magnet. The magnetic resonance device also has a gradient coil. In the direction of the longitudinal axis defined by the main field magnet, the gradient coil has a first side facing away from the main field magnet and a second side facing the main field magnet. The gradient coil has a cross-section with a larger area on the first side facing away than on the second facing side. The gradient coil is provided to generate variable magnetic gradient fields, the gradient fields being provided for magnetic resonance imaging in the imaging region of the magnet volume.

### CROSS-REFERENCE TO RELATED APPLICATIONS

This application claims priority to DE Application No. 102013214880.1, having a filing date of Jul. 30, 2013, the entire contents of which are hereby incorporated by reference.

### FIELD OF TECHNOLOGY

The following relates to a magnetic resonance device with at least one main field magnet and gradient coil.

### BACKGROUND

Magnetic resonance devices are used to generate sectional images of the body of a patient which provide a treatment provider with information about the current anatomical and/or physiological state of the patient.

Standard magnetic resonance devices contain at least one main field magnet, which generates a magnet volume that has an imaging region that is suitable for magnetic resonance imaging. Magnetic resonance devices also contain at least one gradient coil, which generate variable magnetic gradient fields, which are required for the spatial encoding of the magnetic resonance signal.

The imaging region of the magnet volume of conventional magnetic resonance devices that is suitable for magnetic resonance imaging is located in the center of the main field magnet. Therefore, the body region for which magnetic resonance recordings are being produced is generally hard to access from the outside and spatially restricted.

An asymmetric superconducting magnet for magnetic resonance imaging is known from U.S. Pat. No. 6,140,900, having an imaging region located at one end of the magnet. The magnet for magnetic resonance imaging is provided to reduce the claustrophobia some patients experience during a magnetic resonance examination.

### SUMMARY

An aspect relates to a magnetic resonance device, which provides more free space for the patient and treatment providers than a conventional magnetic resonance device.

A further aspect relates to a magnetic resonance device which has at least one main field magnet provided to generate a magnet volume, the magnet volume having an imaging region, with the imaging region being displaced from the center of the main field magnet along a longitudinal axis defined by the main field magnet. The magnetic resonance device also has at least one gradient coil, which in the direction of the longitudinal axis defined by the main field magnet have a first side facing away from the main field magnet and a second side facing the main field magnet, the gradient coil having a cross-section with a larger area on the first side facing away than on the second facing side and the gradient coil being provided to generate variable magnetic gradient fields, the gradient fields being provided for magnetic resonance imaging in the imaging region of the magnet volume.

The longitudinal axis defined by the main field magnet can be an axis of symmetry, in respect of which the magnetic field-generating elements of the main field magnet are arranged symmetrically. The magnet volume here can be configured with axis symmetry, even conically for example, in respect of the longitudinal axis defined by the main field magnet. The magnet volume can be configured with asymmetry in respect of at least one axis which is aligned perpendicular to the longitudinal axis defined by the main field magnet. The magnet volume can be sufficiently homogeneous in the imaging region for it to be used to generate magnetic resonance sectional images of a body region of a patient positioned in the imaging region, said images being able to supply a treatment provider with diagnostic information. The imaging region may even be sufficiently homogenous if it has a homogeneity, or a homogeneity value, greater than 12 ppm, in particular greater than 16 ppm, in particular greater than 20 ppm. For specific applications, a low homogeneity of the imaging region can generally be sufficient, being reflected in a greater homogeneity value. The imaging region of the magnet volume can be a homogeneous region of the magnet volume. The cited homogeneity does not have to be present for the imaging region as a whole. There can be several, in particular more than two, in particular more than four, in particular more than six, possibly even more, subvolumes present, which together may cover the imaging volume as a whole, each having its own cited homogeneity. Specific setting of the gradient fields generated by the gradient coil for the individual subvolumes with their own homogeneity can then produce the homogeneity required for diagnostic magnetic resonance sectional images in the imaging region as a whole. The subvolumes can be cuboidal or can even be configured in the manner of a dish segment based on the magnet characteristics.

The shape of the gradient coil can differ from the usual shape of a cylinder jacket. The shape of the gradient coil here can be defined by the surround of the gradient coil, in which the conductor paths of the gradient coil is integrated. A plurality of gradient coils may be used, and the plurality of gradient coils may be arranged in such a manner that they have a spatially opening configuration in the direction of the longitudinal axis defined by the main field magnet. The opening of the gradient coil can in particular be effected here from the second side facing the main field magnet to the first side facing away from the main field magnet. The first side of the gradient coil here can be further away from the center of the main field magnet along the longitudinal axis than the second side of the gradient coil. The gradient coil here can be arranged with axis symmetry in respect of the longitudinal axis defined by the main field magnet. The gradient coil can thus be arranged asymmetrically in respect of at least one axis, which is aligned perpendicular to the longitudinal axis defined by the main field magnet. The gradient fields generated by the gradient coil may allow spatial encoding of the magnetic resonance signal in the imaging region of the magnet volume in three independent spatial directions.

The combination of a magnet volume with a displaced imaging region and the open configuration of the gradient coil may allow the recording of magnetic resonance sectional images of a body region of a patient positioned in the imaging region of the magnet volume. The main field magnet and the gradient coil may each be optimized in relation to one another here. For example, the magnetic gradient fields generated by the gradient coil can compensate for a lack of homogeneity of the magnet volume in the imaging region or in part of the imaging region.

The present configuration of the magnet volume and gradient coil also may allow simpler access to the body region of the patient being examined. The patient also has more free space.

This makes the magnetic resonance device suitable for recording magnetic resonance sectional images of the pelvic region of a pregnant woman immediately before or during the birth of the baby. This can be advantageous if complications occur for the pregnant woman or the baby during the birth of the baby. Although many risk factors for problems during the birth are detected by means of ultrasound before the birth, ultrasound imaging does not provide a great deal of information during the birth. One reason for this is that bone structures, for example the pelvis, impede propagation of the ultrasound waves. Magnetic resonance imaging of the pelvic region of pregnant women immediately before or during the birth of the baby can therefore be advantageous. The displacement of the imaging region of the magnet volume and the opening arrangement of the gradient coil may allow treating physicians or midwives free access within arm's reach to the pelvis of the woman being examined and provide sufficient free space for the legs of the woman being examined in the birthing position. Even very crude recordings of the woman's pelvis provide very useful diagnostic information during the birthing process or immediately before the birth of a baby, and help the treating physician to plan how to proceed further if problems occur during the birthing process. Therefore, only quite a low homogeneity of the imaging region of the magnet volume is often required for this specific application.

In one embodiment, the main field magnet is configured in such a manner that the magnet volume in the imaging region has at least one static field gradient. The magnet volume can also have just one static field gradient. The magnet volume can also have a number of local static field gradients with possibly different field gradient strengths in subregions of the imaging region. The static field gradient of the magnet volume in the imaging region can be limited. A possible maximum value of the static field gradient of the magnet volume can be 5 mT/m, in particular 10 mT/m, in particular 15 mT/m. The static field gradients of the magnet volume in the imaging region can be compensated for by superimposing gradient fields generated by the gradient coil. Superimposing from the static field gradient with the gradient fields generated by the gradient coil can result in a sufficient cited homogeneity of the magnet volume in the imaging region, which is sufficient for producing diagnostic magnetic resonance sectional images in the imaging region. The magnet volume then may not have to have a cited homogeneity in the imaging region in its own right. A specified gradient field strength of the gradient coil can be reserved to compensate for the static field gradient of the magnet volume in the imaging region. This reserved gradient field strength can then possibly no longer be used for the spatial encoding of the magnetic resonance signal.

In one embodiment, the magnetic resonance device has switchable coils, which are configured in such a manner as to generate non-linear magnetic fields in the imaging region of the magnet volume. The switchable coil can comprise the gradient coils. The gradient coil can then be configured in such a manner that they can also generate in principle unwanted non-linear magnetic fields. The switchable coils can also comprise additional coils that are different from the gradient coil. The switchable coils may not be transmit or receive coils. The switchable coils may not have the purpose of exciting the spins in the imaging region or the purpose of receiving magnetic resonance signals. Instead, the switchable coils can have the purpose of helping to homogenize the magnet volume generated by the main field magnet, in particular, in the imaging region. Non-linear magnetic fields can be higher order magnetic fields. The non-linear magnetic fields can be used to improve the local homogeneity of the magnet volume, in particular, in the imaging region. The field distribution generated by the switchable coils may not be of the gradient type, but can be described, for example, by higher order spherical functions. The switchable coils can allow encoding options, such as PatLoc encoding, O-space encoding or zero-space encoding.

In another embodiment, the gradient coil may have a power input apparatus, the power input apparatus being located on the second side of the gradient coil facing the main field magnet. Magnetic gradient fields are generated, in that current flows in opposite directions through the conductor paths of the gradient coil on two opposing sides in respect of the imaging region of the magnet volume. A greater gradient field strength can be present in the region enclosed by the conductor paths of the gradient coil than in the region in which the power is input into the conductor paths of the gradient coil. The arrangement of the conductor paths of the gradient coil according to this embodiment therefore may result in an increased gradient field strength in the imaging region of the magnet volume and therefore an improved image quality. The location of the power input apparatus on the side facing the main field magnet can mean that the gradient field strength is only reduced in a body region of the patient that is not to be examined using the magnetic resonance device.

According to one embodiment, the main field magnet is configured in such a manner that a tunnel-type opening results in the center of the main field magnet, in which a patient can be positioned essentially along the longitudinal axis defined by the main field magnet, and the imaging region of the magnet volume is located at one end of the tunnel-type opening. The magnetic field-generating elements of the main field magnet here can be arranged in the manner of a hollow cylinder around the patient, resulting in the tunnel-type opening, in which the patient can be positioned. The displacement of the imaging region of the magnet volume to one end of the tunnel-type opening means that the body region of the patient being examined by the magnetic resonance device can also be positioned at one end of the tunnel-type opening. This provides better accessibility to the body region of the patient being examined from the outside and more free space in respect of the body region of the patient adjoining the body region being examined on the side facing away from the main field magnet. When the magnetic resonance device is used during the birthing process, the upper body of the pregnant woman can be positioned within the tunnel-type opening and the pelvis of the pregnant woman is located at one end of the tunnel-type opening. The legs of the pregnant woman are then largely located outside the tunnel-type opening. The patient support apparatus can be embodied as a patient table, it being possible to adjust the position of the patient table.

In a further embodiment, the main field magnet is configured in such a manner that part of the imaging region of the magnet volume is located outside the tunnel-type opening. This configuration of the main field magnet further improves accessibility to the body region of the patient being examined and provides more free space directly for the body region of the patient being examined. The reduction of the homogeneity of the imaging region caused by the displacement of the imaging region of the magnet volume can also be sufficient for specific applications, such as the recording of magnetic resonance sectional images during the birthing process for example. The reduction of the homogeneity of the imaging region can be compensated for as described to some degree by gradient fields and/or higher order magnetic fields, which are generated by the gradient coil and/or switchable coil.

In one exemplary embodiment, the gradient coil is configured in the manner of a jacket of a truncated cone, the axis of symmetry of which runs parallel to the longitudinal axis defined by the main field magnet. In particular, the axis of symmetry of the truncated cone can correspond to the longitudinal axis defined by the main field magnet. The surround of the gradient coil, in which the conductor paths of the gradient coil is integrated, is in turn configured in the manner of the jacket of a truncated cone. The truncated cone may be open from the side facing the main field magnet to the side facing away from the main field magnet. The imaging region of the magnet volume may therefore be within the volume defined by the truncated cone. This arrangement of the gradient coil may result in better accessibility from the side facing away from the main field magnet to the body region of the patient being examined and may provide more free space in respect of the body region of the patient being examined, in particular, in respect of any direction perpendicular to the longitudinal axis defined by the main field magnet.

In a further embodiment, the magnetic resonance device may have a patient support apparatus, the gradient coil having a lower coil segment located below a patient support apparatus and the gradient coil having an upper coil segment located above the patient support apparatus, the lower coil segment having a smaller curvature in cross-section than the upper coil segment. The flattening of the lower coil segment of the gradient coil can reduce the distance between the lower coil segment of the gradient coil and the patient. This may increase the gradient field strength in the body region of the patient being examined and therefore may also improve the performance of the gradient system, as the magnetic field generated by the gradient coil decreases significantly as the distance increases. The upper coil segment of the gradient coil can also be configured so that it opens in the manner of part of a jacket of a truncated cone.

In a further embodiment, the magnetic resonance device may have a patient support apparatus, with most, in particular all, of the main field magnet being arranged below the patient support apparatus. This embodiment may contain an arrangement of the main field magnet that is fundamentally different from the embodiments described above. The patient support apparatus here may have a first side, on which a patient can be positioned. The patient support apparatus has a second side opposite the first side. Most, in particular all, of the main field magnet is then arranged on the second side of the patient support apparatus. The patient can therefore be positioned radially to a longitudinal axis defined by the main field magnet. The body region of the patient being examined can then be positioned directly above the center of the main field magnet. This arrangement of the main field magnet may mean that accessibility to the body region being examined from the front and side and the free space for the patient are in no way restricted by the main field magnet. The patient support apparatus can be configured in such a manner that it can be positioned quickly on the main field magnet and removed again quickly from above or from the side.

In one exemplary embodiment, the main field magnet may have a recess positioned on the side of the main field magnet facing toward the patient support apparatus, the gradient coil on the second side of the gradient coil facing the main field magnet being let into the recess in the main field magnet. This arrangement of the gradient coil may relate to the embodiment in which most, in particular all, of the main field magnet is arranged below the patient support apparatus. Together with the main field magnet and the patient support apparatus, this arrangement of the gradient coil can result in optimum utilization of the available space.

In a further embodiment, the gradient coil is configured in the shape of a dish, the base of the dish being located on the second side of the gradient coil facing the main field magnet and the dish being open in the direction of the patient support apparatus. The base of the dish can be integrated in the recess in the main field magnet. The patient can be positioned in the dish formed by the gradient coil. The dish walls may therefore be positioned to the sides of the patient. This arrangement of the gradient coil may allow the generation of sufficiently powerful and linear gradient fields, which are suitable for magnetic resonance imaging in the imaging region of the magnet volume. Accessibility from above to the body region of the patient being examined can also be ensured.

Embodiments of the magnetic resonance device can also contain high-frequency antennas, which are provided to transmit excitation pulses and receive magnetic resonance signals specifically in the imaging region of the magnet volume. The high-frequency antennas can be flexible and movable, so that it is possible to remove the high-frequency antennas from the body region of the patient being examined if required.

### BRIEF DESCRIPTION

Some of the embodiments will be described in detail, with reference to the following figures, wherein like designations denote like members, wherein:

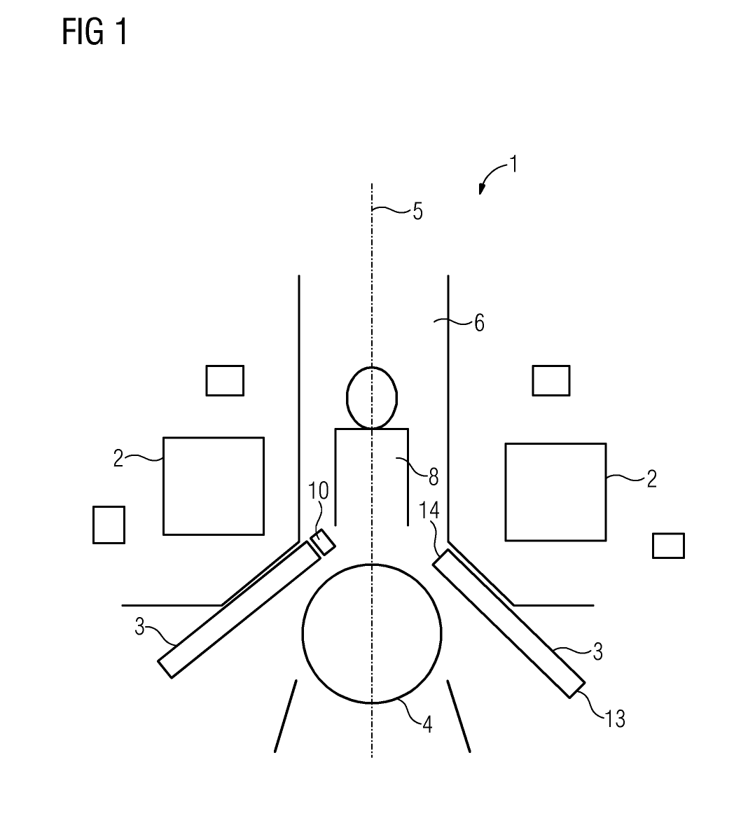

FIG. 1 shows a plan view of a first embodiment of a magnetic resonance device;

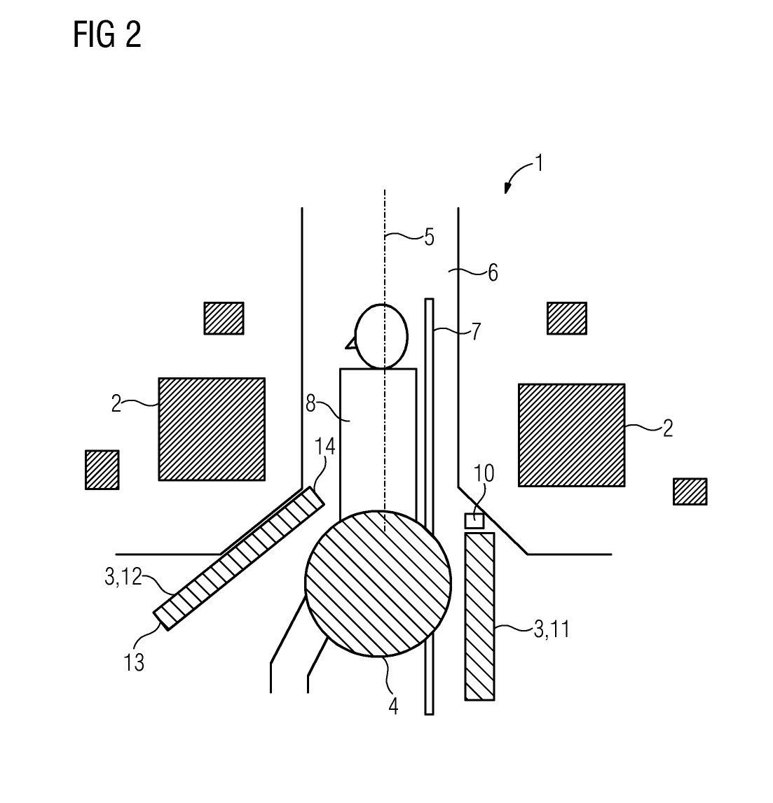

FIG. 2 shows a longitudinal section through the first embodiment of the magnetic resonance device;

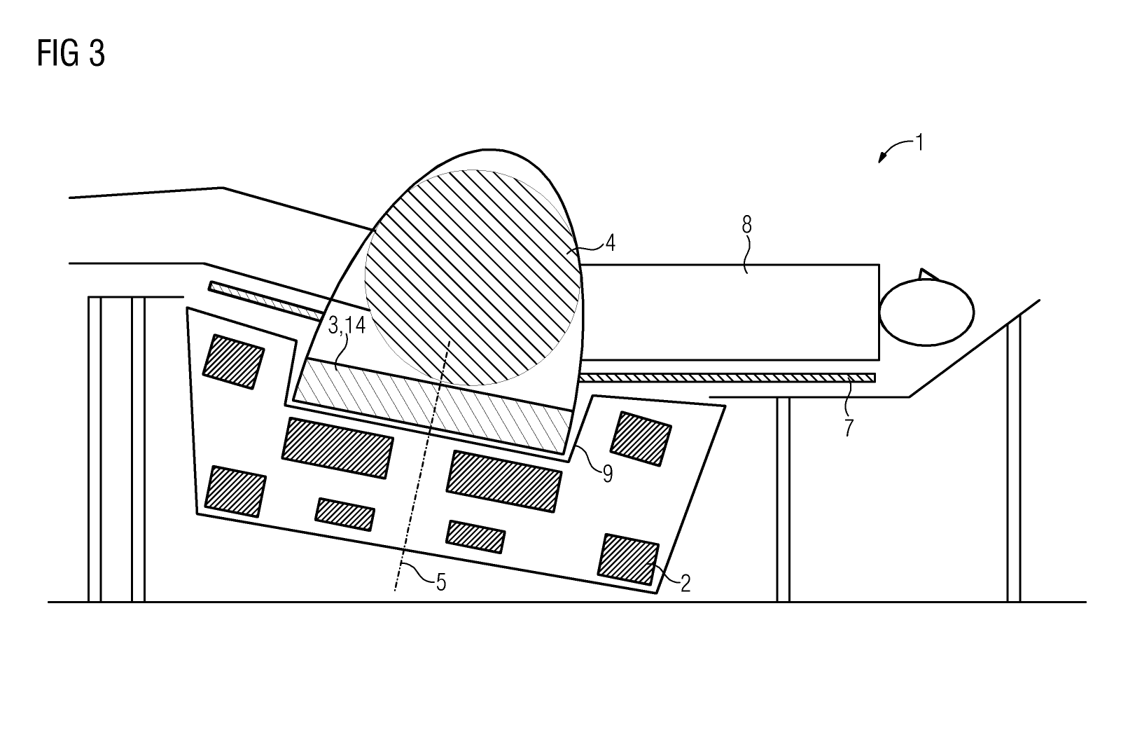

FIG. 3 shows a longitudinal section through a second embodiment of a magnetic resonance device; and

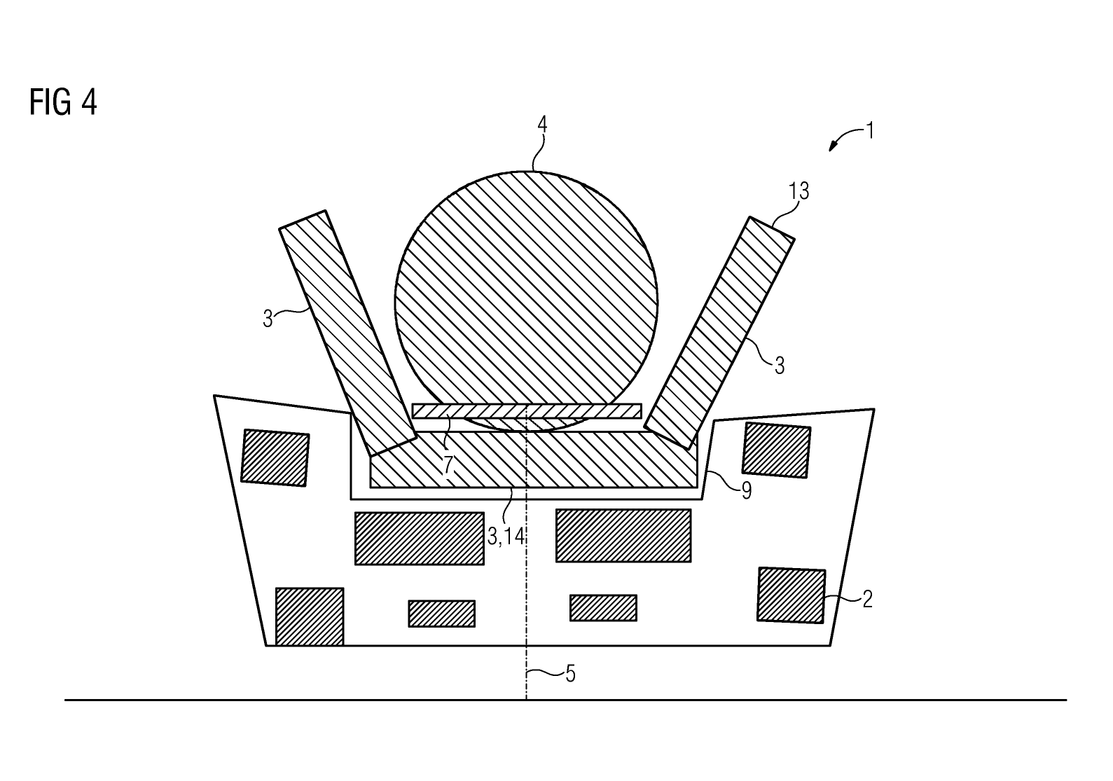

FIG. 4 shows a cross-section through the second embodiment of the magnetic resonance device shown in FIG. 3.

### DETAILED DESCRIPTION

FIG. 1 shows a plan view of a first embodiment of a magnetic resonance device 1. The magnetic resonance device 1 has a main field magnet 2, which is provided to generate a magnet volume. The magnet volume has an imaging region 4, in which the body region of a patient 8 to be examined by the magnetic resonance device 1 is positioned. The main field magnet 2 has a number of magnetic field-generating elements, which are arranged in the manner of a hollow cylinder around the patient 8. This results in a tunnel-type opening 6 having a longitudinal axis 5. The magnetic resonance device 1 also has at least one gradient coil 3 in a spatially opening configuration.

The magnetic field-generating elements of the main field magnet 2 are arranged in such a manner that the imaging region 4 of the magnet volume is displaced along the longitudinal axis 5 defined by the main field magnet 2 to one end of the tunnel-type opening 6. Part of the imaging region 4 of the magnet volume is located outside the tunnel-type opening 6. FIG. 1 shows an exemplary arrangement of the magnetic field-generating elements of the main field magnet 2, which results in at least one desired displacement of the imaging region 4 of the magnet volume. However, a different arrangement of the magnetic field-generating elements of the main field magnet 2 can also be used. Displacement of the imaging region 4 of the magnet volume to the physical end of the tunnel-type opening 6 of the main field magnet 2 allows treatment providers direct access, in particular, with their hands, to a body region of the patient 8 to be examined by the magnetic resonance device 1 and positioned for this purpose in the imaging region 4.

The main field magnet 2 is in turn configured in such a manner that its shape is tailored to the shape of the gradient coil 3. If the configuration calls for a plurality of gradient coils, some or all of the gradient coil(s) 3 are arranged in the manner of a jacket of a truncated cone, enclosing the imaging region 4 of the magnet volume. The lower segment of the gradient coil(s) 3 is not shown in FIG. 1. It can be flattened as shown in FIG. 2 or it can follow the jacket of the truncated cone. The tunnel-type opening 6 therefore has a chamfer at one end, providing space for the truncated cone. The power input into the gradient coil is brought about by a power input apparatus 10, which is located on the side 14 of the gradient coil 3 facing the main field magnet 2. The power input apparatus 10 shown is only outlined schematically here. This arrangement of the gradient coil(s) 3 allows variable magnetic gradient fields to be generated, which are tailored to the magnet volume generated by the main field magnet 2 in such a manner that they are suitable for recording magnetic resonance sectional images in the imaging region 4 of the magnet volume. The arrangement of the gradient coil(s) 3, which opens in a tapering manner toward the foot end of the patient table 5, improves accessibility to the body region of the patient 8 being examined from the front. The illustrated arrangement of the gradient coil(s) 3 also increases the free space for the patient 8.

This exemplary embodiment of the magnetic resonance device 1 can be particularly suitable for recording magnetic resonance sectional images in the pelvic region of a pregnant woman immediately before or during the birth. In the instance shown therefore, the pelvic region of the patient 8 is positioned in the imaging region 4 of the magnet volume. Sufficient free space is provided for the legs of the patient 8 by the opening of the gradient coil(s) 3. In the instance shown, the pelvic region of the patient 8 can be accessed from the front within arm's reach. The upper body of the patient 8 is located within the tunnel-type opening 6.

FIG. 2 shows a longitudinal section through the first embodiment of the magnetic resonance device 1. It shows a particular arrangement of the gradient coil(s) 3, which are flattened on the patient support apparatus 7 side. The lower coil segment 11 of the gradient coil(s) is less curved in cross-section than the upper coil segment 12 of the gradient coil(s) 3. In the instance shown, the lower coil segment 11 is configured as flat. The upper coil segment 12 is also open in the direction of the side 13 of the gradient coil(s) 3 facing away from the main field magnet 2.

This reduces the distance between the flattened lower coil segment 11 of the gradient coil(s) 3 and the body region of the patient 8 being examined. This increases the gradient field strength in the body region of the patient 8 being examined and therefore also improves the performance of the gradient system, as the magnetic field generated by the gradient coil(s) 3 decreases significantly as the distance increases. Accessibility to the body region of the patient 8 being examined is also ensured due to the opening of the upper coil segment 12 of the gradient coil(s) 3.

FIG. 3 shows a longitudinal section through an alternative second embodiment of the magnetic resonance device 1. All of the main field magnet 2 is arranged below the patient support apparatus 7. The main field magnet 2 therefore does not restrict accessibility to the body region of the patient 8 being examined from above or from the side.

In this exemplary embodiment, the body region of the patient 8 being examined is positioned directly above the center of the main field magnet 2. The patient 8 is positioned essentially radially to a longitudinal axis 5 defined by the main field magnet 2. However, the patient 8 can also be positioned differently. In particular, a magnetic resonance device 1 shown in this exemplary embodiment can examine any body part of a patient 8, providing the patient 8 with more free space than conventional magnetic resonance devices.

In the instance shown, the gradient coil 3 is configured in the manner of a dish, the base of the dish being let into a recess 9 in the main field magnet 2. The gradient coil 3 is also arranged in such a manner that they enclose the imaging regions 4 of the magnet volume. A different shape for the gradient coil 3, which allows the treatment providers access to the the body region of the patient 8 being examined, is also possible.

In the instance shown, the patient support apparatus 7 is tailored to the shape of the main field magnet 2 and the patient 8. It is therefore optimized for recording magnetic resonance sectional images of the pelvic region. The patient support apparatus 7 is also embodied in such a manner that it can be removed in a simple manner from the magnetic resonance device 1, and can be positioned in a simple manner on the magnetic resonance device 1. An embodiment of the patient support apparatus 7 as a movable patient table is also possible.

FIG. 4 shows a cross-section through the second embodiment of the magnetic resonance device 1 shown in FIG. 3. The gradient coil(s) 3 shown are arranged here so that they open in the manner of a dish. The base of the dish is located here on the side 14 of the gradient coil(s) 3 facing the main field magnet 2 and let into a recess 9 in the main field magnet 2. The sides of the dish are to the sides of the patient support apparatus 7. The sides of the dish can also be arranged flatter than shown, improving accessibility to the body region of the patient 8 being examined from the side even further.

The exemplary embodiment of the magnetic resonance device 1 shown in FIG. 3 and FIG. 4 can also be particularly suitable for recording magnetic resonance sectional images in the pelvic region of a pregnant woman immediately before or during the birth.

Even though the invention was illustrated and described in detail using exemplary embodiments, the invention is not restricted by the disclosed examples and other variations can be derived therefrom by the person skilled in the art without departing from the scope of protection of the invention.

To summarize, the embodiments of the invention relate to a magnetic resonance device 1 with at least one main field magnet 2 and at least one gradient coil 3. So that embodiments of the magnetic resonance device 1 provides more free space for the patient 8 and treatment providers, at least one exemplary embodiment of the magnetic resonance device 1 has at least one main field magnet 2, which is provided to generate a magnet volume. The magnet volume has an imaging region 4, which is displaced from the center of the main field magnet 2 along a longitudinal axis 5 defined by the main field magnet 2. The magnetic resonance device 1 also has at least one gradient coil 3. In the direction of the longitudinal axis 5 defined by the main field magnet 2 the gradient coil 3 have a first side 13 facing away from the main field magnet 2 and a second side 14 facing the main field magnet 2. The gradient coil therefore has a cross-section with a larger area on the first side 13 facing away than on the second facing side 14. The gradient coil 3 is provided to generate variable magnetic gradient fields, the gradient fields being provided for magnetic resonance imaging in the imaging region 4 of the magnet volume.

1. A magnetic resonance device comprising:
at least one main field magnet, which is provided to generate a magnet volume, the magnet volume having an imaging region, the imaging region being displaced from a center of the main field magnet along a longitudinal axis defined by the main field magnet; and
at least one gradient coil, which in a direction of the longitudinal axis defined by the main field magnet has a first side facing away from the main field magnet and a second side facing the main field magnet, the gradient coil having a cross-section with a larger area on the first side facing away from the main field magnet than on the second side facing the main field magnet;
wherein the gradient coil is provided to generate variable magnetic gradient fields, the variable magnetic gradient fields being provided for magnetic resonance imaging in the imaging region of the magnet volume.

2. The magnetic resonance device as claimed in claim 1, wherein the main field magnet is configured in such a manner that the magnet volume has at least one static field gradient in the imaging region.

3. The magnetic resonance device as claimed in claim 1, further comprising a plurality of switchable coils configured in such a manner as to generate non-linear magnetic fields in the imaging region of the magnet volume.

4. The magnetic resonance device as claimed in claim 1, wherein the at least one gradient coil has a power input apparatus, the power input apparatus being located on the second side of the at least one gradient coil facing the main field magnet.

5. The magnetic resonance device as claimed in claim 1, wherein the main field magnet is configured in such a manner that a tunnel-type opening results in the center of the main field magnet, in which a patient is positioned essentially along the longitudinal axis defined by the main field magnet, and the imaging region of the magnet volume is located at one end of the tunnel-type opening.

6. The magnetic resonance device as claimed in claim 5, wherein the main field magnet is configured in such a manner that part of the imaging region of the magnet volume is located outside the tunnel-type opening.

7. The magnetic resonance device as claimed in claim 5, wherein the at least one gradient coil is configured in a manner of a jacket of a truncated cone, an axis of symmetry of which runs parallel to the longitudinal axis defined by the main field magnet.

8. The magnetic resonance device as claimed in claim 5, further comprising a patient support apparatus, the at least one gradient coil having a lower coil segment located below the patient support apparatus, and the at least one gradient coil having an upper coil segment located above the patient support apparatus, the lower coil segment having a smaller curvature in cross-section than the upper coil segment.

9. The magnetic resonance device as claimed in claim 1, further comprising a patient support apparatus, with at least one of most and all of the main field magnet being arranged below the patient support apparatus.

10. The magnetic resonance device as claimed in claim 9, wherein the main field magnet has a recess positioned on a side of the main field magnet facing toward the patient support apparatus, the at least one gradient coil on the second side facing the main field magnet being let into the recess in the main field magnet.

11. The magnetic resonance device as claimed in claim 9, wherein the at least one gradient coil is configured in a shape of a dish, a base of the dish being located on the second side facing the main field magnet and the dish being open in a direction of the patient support apparatus.
